(19) United States
(12) Patent Application Publication
(10) Pub. No.: US 2019/0192252 A1
(43) Pub. Date: Jun. 27, 2019

(54) Surgical Instrument Positioning System and Positioning Method Thereof

(71) Applicant: EPED Inc., Kaohsiung City (TW)

(72) Inventors: Ta-Ko Huang, Kaohsiung City (TW); Jerry T. Huang, Kaohsiung City (TW)

(21) Appl. No.: 16/218,005

(22) Filed: Dec. 12, 2018

(30) Foreign Application Priority Data
Dec. 27, 2017 (CN) 106146044
Jul. 12, 2018 (CN) 107124148

(51) Int. Cl. A61B 90/00; A61B 90/50

(52) U.S. Cl. CPC A61B 90/37; A61B 90/361; A61B 90/90; A61B 2090/373; A61B 2090/3762; A61B 90/50

(57) ABSTRACT

A surgical instrument positioning system and a positioning method thereof are provided. The natural feature information datum a of the human body presented by a medical image (for example, CT) taken before surgery is first input into the positioning system, and 3D datum reconstruction is performed. The position information of the hand-held instrument, obtained by the light sensing element, is then transmitted back to the positioning system. Then, the feature points can be automatically suggested via the positioning system or the feature points can be automatically designated or the feature points can be manually selected by the operator to establish the coordinate relationship between the patient position and the hand-held instrument, thereby solving the problem of the impact of the prior art on the surgical operation.

### FIELD OF THE INVENTION

The present invention relates to a surgical instrument positioning system and a positioning method thereof used for a surgical operation.

### BACKGROUND OF THE INVENTION

For performing precision surgery, the position for the scalpel to dissect or puncture is one of the important factors for successful surgery. Therefore, when such surgery is performed, the position of the surgical needle is confirmed through image guidance, thereby avoiding the error caused by the distance difference in the position of the surgery and the influence of the surgical result. Common applications for such surgery include vertebral screw placement, live sampling, spinal nerve block, or tooth implantation. In one of the conventional surgical techniques, in order to ensure the position of the instrument, the operator needs to repeatedly photograph with X-ray to guide the advancement of the needle to confirm the final position of the needle. The precise placement of the needle provides a guide path for subsequent surgical instruments, thereby achieving precise treatment. However, the aforementioned invasive surgery may utilize an external guidance system (or navigation system) to record or inform the operator of the current position of the surgical instrument.

One of the guiding methods is to attach a reflective element to a surgical instrument. Before the operation is performed, the surgical instrument is registered in the guiding system to confirm the relative positional relationship between the reflective element and the surgical instrument. During the operation, a plurality of reflective elements is provided at the surgical site (on the human body) for the guiding system to analyze the relative positional relationship between the surgical instrument and the surgical site. However, the method of arranging the reflective elements at the surgical site may easily form obstacles during the operation, for example, affecting the movement path of the instrument. In view of the drawbacks of the prior art, the present invention is to solve the inconvenience and difficulty in the conventional surgical guiding system. In view of the above problems, the present invention provides a surgical instrument positioning system and a positioning method thereof to solve the aforementioned shortcomings.

### SUMMARY OF THE INVENTION

The present invention mainly uses a certain position of the human body as a feature point, instead of the prior art (a reflective element is disposed at a corresponding portion). Thereby, the problems of the prior art can be overcome. The primary object of the present invention is to provide a surgical instrument positioning system and a positioning method thereof. The method according to the present invention is based on analyzing the surface image datum of the surgical site or its surroundings. The operator specifies or selects at least three positions, or the at three positions may be suggested or selected by the system to be correspondingly set and recorded as a first coordinate datum, a second coordinate datum and a third coordinate datum. The first coordinate datum, the second coordinate datum and the third coordinate datum are established as a first feature point, a second feature point and a third feature point in the positioning system. The first feature point, the second feature point and the third feature point may be set as absolute positions. The electronic computer is equipped with a surgical instrument display software. The relative position of a real-time image position datum of a light sensing element is calculated by the datum returned by the light sensing element and the first coordinate datum, the second coordinate datum and the third coordinate datum, and is then imaged on the screen. Through the invention, it is not necessary to provide a plurality of reflective elements at or around the surgical site to overcome the obstacles caused by reflective elements during the operation.

According to one aspect of the present invention, the present invention provides a surgical instrument positioning system used for confirming the position of a front end of a hand-held instrument relative to a surgical site inside a human body when performing a surgical operation. The surgical instrument positioning system comprises a light sensing element, an image capturing device, an electronic computer, and a display device. The light sensing element is disposed on the hand-held instrument. The image capturing device is configured to continuously capture a surface image datum of the surgical site of the human body and a real-time image position datum of the light sensing element.

The electronic computer is electrically connected to the image capturing device for receiving the surface image datum and the real-time image position datum of the light sensing element. The electronic computer calculates the position of the hand-held instrument relative to the human body and the image position datum based on the position of the light sensing element. The electronic computer is equipped with a surgical instrument display software for analyzing the surface image datum to find at least three suitable positions to be correspondingly set and recorded as a first coordinate datum, a second coordinate datum and a third coordinate datum. The suitable positions may be directly specified by the user or suggested to the user after analysis. The first coordinate datum, the second coordinate datum and the third coordinate datum are established as a first feature point, a second feature point and a third feature point, respectively. The display device is electrically connected to the electronic computer for displaying the first feature point, the second feature point and the third feature point and a real-time simulated image of the hand-held instrument.

In the aforesaid he surgical instrument positioning system, the first coordinate datum, the second coordinate datum and the third coordinate datum are absolute positions. The real-time image position datum of the light sensing element is a relative position of the first coordinate datum and the second coordinate datum and the third coordinate datum.

Preferably, the surgical instrument positioning system further comprises an input device for inputting the first coordinate datum and the second coordinate datum and the third coordinate datum. The input device is electrically connected to the electronic computer. The input device being configured to select the first feature point or the second feature point or the third feature point by the surgical instrument display software displayed by the display device, the surgical instrument display software confirming whether the hand-held instrument on which the light sensing element is mounted reaches the position of the first feature point or the second feature point or the third feature point through the image capturing device.

The user can manipulate the input device in cooperation with the content presented by the display device for selecting the first feature point, the second feature point and the third feature point to be input into the surgical instrument positioning system. The surgical instrument can directly select a feature point or automatically scan (line-to-line or plane-to-plane) to achieve the purpose of positioning. The surgical instrument display software can confirm whether the light sensing element reaches the corresponding position through the image capturing device.

In order to accurately calculate the positioning information required by the system, the first feature point, the second feature point and the third feature point may be a human body feature. The human body feature has a partially identified image, such as a canthus, a nasal tip, an interdental gap, a tooth line trend or a tooth socket.

According to another aspect of the present invention, the present invention provides a surgical instrument positioning method used for confirming the position of a front end of a hand-held instrument relative to a surgical site inside a human body when performing a surgical operation. The surgical instrument positioning method comprises the following steps: (A) providing a light sensing element on the hand-held instrument; (B) providing an image capturing device for continuously or intermittently capturing a surface image datum of the human body and a real-time image position datum of the light sensing element; (C) calculating the position of the hand-held instrument relative to the human body and the image position datum based on the surface image datum and the position of the light sensing element; (D) providing a surgical instrument display software for analyzing at least three positions of the surface image datum to be correspondingly set and recorded as a first coordinate datum, a second coordinate datum and a third coordinate datum, the first coordinate datum, the second coordinate datum and the third coordinate datum being respectively established as a first feature point, a second feature point and a third feature point; and (E) providing a display device for displaying the first feature point, the second feature point, the third feature point, and the real-time position of the hand-held instrument or a simulated image.

Preferably, the surgical instrument positioning method further comprises the following steps after the step (E): providing an input device for selecting the first feature point or the second feature point or the third feature point, the surgical instrument display software displayed by the display device displaying the image capturing device and confirming whether the light sensing element reaches the position of the first feature point or the second feature point or the third feature point through the image capturing device.

Preferably, a calculation method of the surgical instrument display software comprises: (a) loading a medical image; (b) processing the medical image; (c) reconstructing a 3D datum; (d) determining a feature datum; (e) obtaining a feature datum of an actual space; (f) obtaining an initial alignment matrix or (f-1) performing a feature point correspondence algorithm to obtain an transformation matrix of the image and the actual space; (g) obtaining a surface datum of the actual space; (h) performing a surface correspondence algorithm to obtain the transformation matrix of the image and the actual space.

Preferably, the determining in the step (d) of the calculation method is an automatic determining or a manual determining. Preferably, the feature datum in the step (d) of the calculation method is a point, a plane or a part.

Preferably, the feature datum of the actual space in the step (g) of the calculation method is obtained in a contact manner or a non-contact manner.

When the determining in the step (d) of the calculation method is an automatic determining, the process of automatically determining the feature datum comprises: (d-1) loading the medical image; (d-2) image filtering, de-noising and enhancement; (d-3) histogram analysis and image frequency analysis; (d-4) data grouping and condition setting; (d-5-1) region growing according to different thresholds, ranges and frequencies; (d-5-2) region growing or dividing according to an anatomical structure; (d-6) calculating gradient and divergence; (d-7) determining a feature part; (d-8) outputting the coordinate, shape and type of the feature part.

It can be seen from the above description that the surgical instrument positioning system and the positioning method thereof of the present invention can achieve the goal as desired. Through the biological characteristic of the patient as an absolute positioning position to cooperate with the light sensing element on the hand-held instrument, there is no need to provide a plurality of reflective elements at the surgical site, thereby solving the obstacles caused by the reflective elements during the operation.

### BRIEF DESCRIPTION OF THE DRAWINGS

FIG. 1 is a schematic view of a surgical instrument positioning system in accordance with the present invention;

FIG. 2 is a flow chart of a surgical instrument positioning method in accordance with the present invention;

FIG. 3 is another flow chart of the surgical instrument positioning method in accordance with the present invention;

FIG. 4 illustrates a feature information registration way of the surgical instrument positioning method in accordance with the present invention; and

FIG. 5 illustrates a flow chart of automatically determining a feature datum of the surgical instrument positioning method of the present invention.

### DETAILED DESCRIPTION OF THE PREFERRED EMBODIMENTS

The present invention relates to a surgical instrument positioning system and a positioning method thereof. Embodiments of the present invention will now be described, by way of example only, with reference to the accompanying drawings.

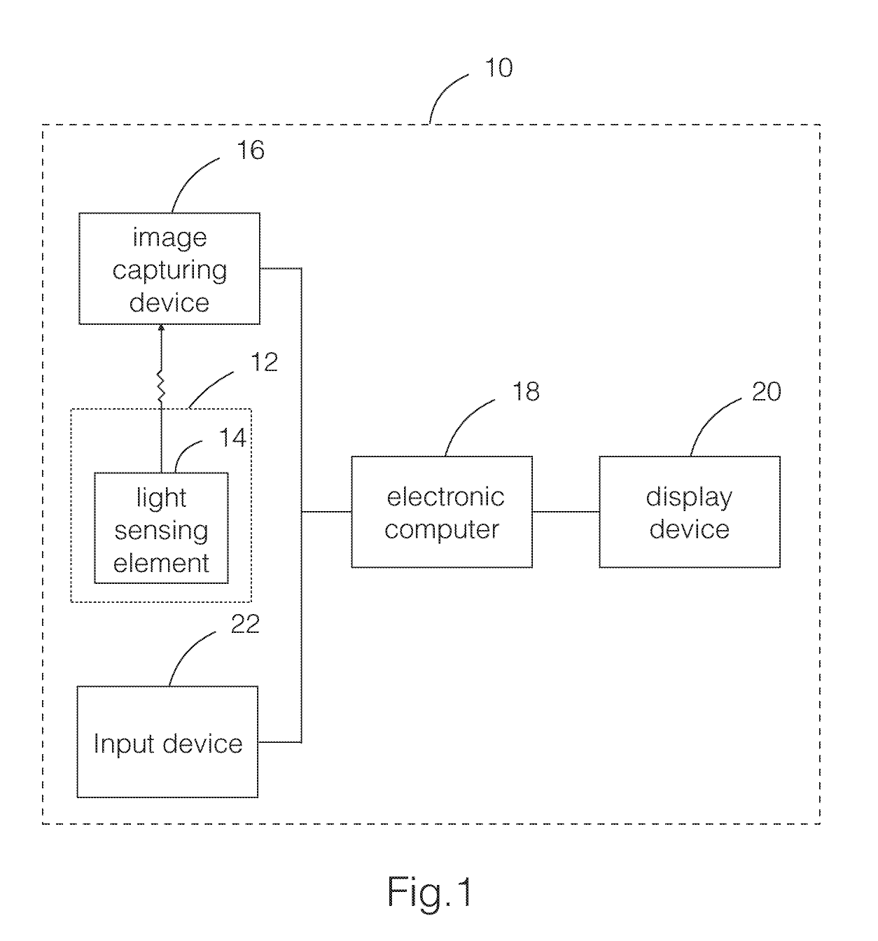

First, please refer to FIG. 1, which is a schematic view of a surgical instrument positioning system in accordance with the present invention. As shown in the figure, a surgical instrument positioning system 10 of the present invention, when performing a surgical operation, is used to confirm the position of a front end of a hand-held instrument 12 relative to a surgical site inside a human body. Wherein, the surgical instrument positioning system 10 comprises a light sensing element 14, an image capturing device 16, an electronic computer 18, and a display device 20.

As described above, the light sensing element 14 of the present invention is disposed on the hand-held instrument 12. The image capturing device 16 is configured to continuously or intermittently capture a partial surface image datum of the human body and a real-time image position datum of the light sensing element 14. The electronic computer 18 is electrically connected to the image capturing device 16 for receiving the surface image datum and the real-time image position datum of the light sensing element 14.

Next, the electronic computer 18 calculates the position of the hand-held instrument 12 based on the position of the light sensing element 14. The electronic computer 18 is equipped with a surgical instrument display software, and can analyze the surface image datum and correspondingly set and record three positions as a first coordinate datum, a second coordinate datum and a third coordinate datum. The first coordinate datum, the second coordinate datum and the third coordinate datum are established as a first feature point, a second feature point and a third feature point, respectively. The display device 20 is electrically connected to the electronic computer 18 for displaying the first feature point, the second feature point and the third feature point and the real-time position of the hand-held instrument 12 or a 3D simulation environment.

For the surgical instrument positioning system 10 as described above, the first coordinate datum, the second coordinate datum and the third coordinate datum are absolute positions. The real-time image position datum of the light sensing element 14 is a relative position of the first coordinate datum and the second coordinate datum and the third coordinate datum.

In addition, the electronic computer 18 of the present invention may be in communication with an input device 22. The input device 22 first selects the first feature point, the second feature point and the third feature point in the system through the simulated image displayed by the display device 20 and confirms the setting for the system to record the relationship of the relative positions of the feature points. The surgical instrument display software can confirm whether the hand-held instrument 12 on which the light sensing element 14 is mounted reaches the position of the first feature point, the second feature point or the third feature point through the image capturing device 16.

Furthermore, for the surgical instrument positioning system 10 disclosed in the present invention, the hand-held instrument 12 is a surgical probe; the electronic computer 18 may be a computer, a portable computer or a tablet computer; the display device 20 may be a screen; the light sensing element 14 may be a reflective element or an active light emitting element; and the input device 22 is a mouse or a touchpad. Furthermore, the first feature point, the second feature point and the third feature point may be human body features, including a canthus, a nasal tip, an interdental gap, or a tooth socket. The first feature point, the second feature point and the third feature point may be established according to a human bone structure. The human bone structure may be a single tooth shape, a tooth line trend, or a tooth socket position.

According to the technical content disclosed in the present invention, under the introduction of the function of the feature point selection, the surgical instrument positioning system 10 may be applied to tooth implantation, without the need to wear a biteplate when performing a tomography scan for optical registration.

The operator can perform a feature point selection according to his/her experience and preferences and select obvious human body features, such as a canthus, a nasal tip, an interdental gap, a tooth socket, a tip of a tooth surface, and the like. The system can record the sequence of feature points selected by the physician and the visual angle of the software when clicking. When the surgical instrument is used to select the actual position, the surgical instrument display software can help guide the operator to the position for selection. Of course, the operator may use automatic scanning (line-to-line or plane-to-plane) to complete the job of selection.

Different regions may be in cooperation with different feature analysis, such as teeth, determining the position of the tooth socket or the position having obvious point, line or plane features according to their shapes, line trend, etc.; or determining the adjacent spacing relationship and further searching for the characteristics of the gap between the teeth according to its boundary characteristics. After analyzing the surface image datum, the software operation can find the position that is most suitable as the feature point to be presented on the display device for reference by the operator. Alternatively, the light spot may be made by infrared rays around the site to be treated, and the light spot is kept in a position adjacent to the site to be treated, and can also be used as a feature point.

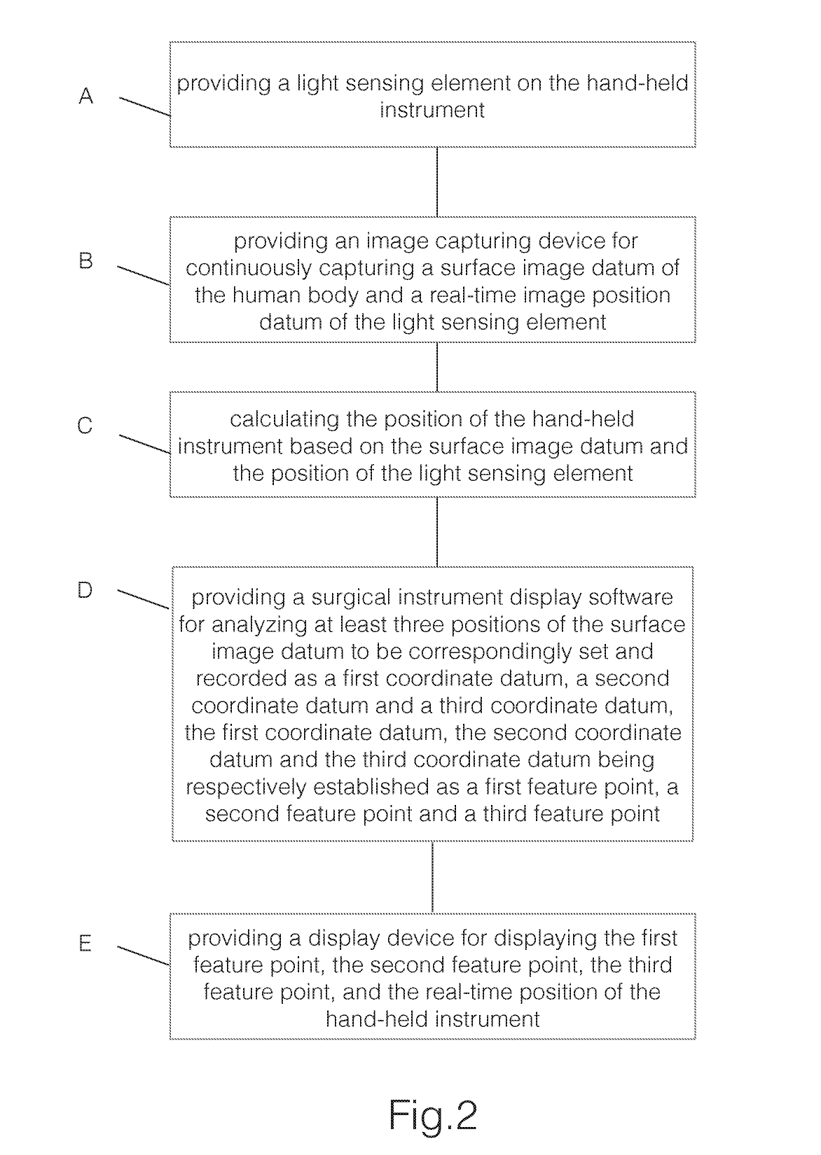

According to the technical presentation of the embodiment of the above system, the present invention may further exemplify an embodiment of a method. Referring to FIG. 2, which is a flow chart illustrating a surgical instrument positioning method of the present invention. As shown in the figure, a surgical instrument positioning method of the present invention can confirm the position of a front end of a hand-held instrument relative to the human body during the surgical operation. The surgical instrument positioning method comprises the following steps. As shown in step (A), a light sensing element is provided on the hand-held instrument. As shown in step (B), an image capturing device is provided for continuously capturing a surface image datum of the human body and a real-time image position datum of the light sensing element. As shown in step (C), the position of the hand-held instrument is calculated based on the surface image datum and the position of the light sensing element.

Then, as shown in step (D), a surgical instrument display software is provided, and the software is used to analyze at least three positions of the surface image datum to be correspondingly set and recorded as a first coordinate datum, a second coordinate datum and a third coordinate datum. The first coordinate datum, the second coordinate datum and the third coordinate datum are respectively established as a first feature point, a second feature point and a third feature point. Finally, as shown in step (E), a display device is provided for displaying the first feature point, the second feature point and the third feature point and the real-time position of the hand-held instrument.

For the surgical instrument positioning method as described above, the first coordinate datum, the second coordinate datum and the third coordinate datum are absolute positions. The real-time image position datum of the light sensing element is a relative position of the first coordinate datum and the second coordinate datum and the third coordinate datum.

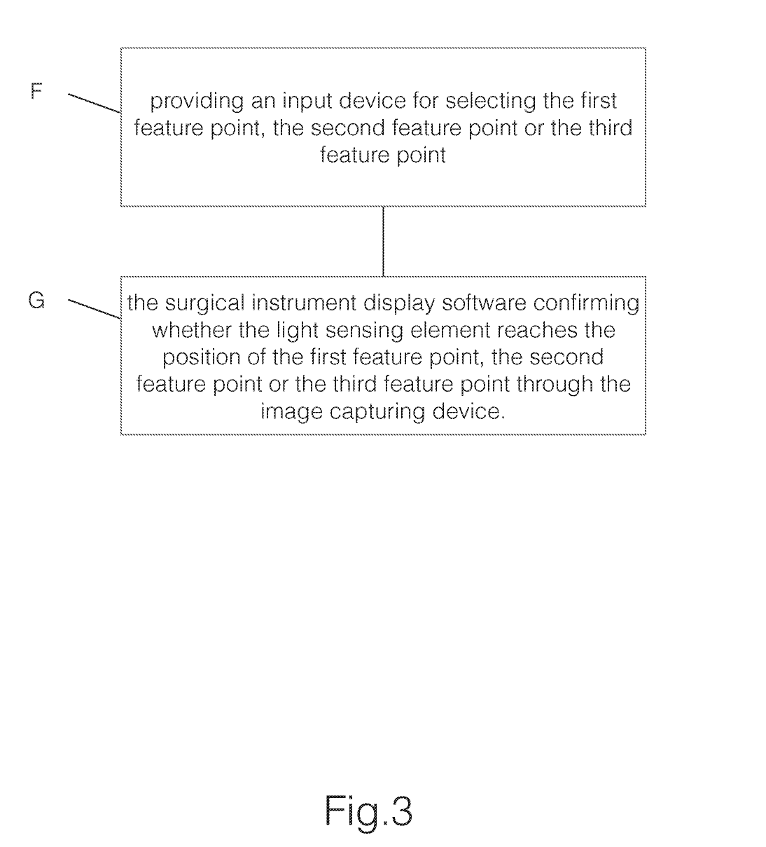

Next, referring to FIG. 3, another flow chart of the surgical instrument positioning method of the present invention is illustrated. As shown in the figure, the surgical instrument positioning method disclosed in the present invention, after the step (E), further comprises the following steps. As shown in step (F), an input device is provided to select the first feature point, the second feature point or the third feature point or automatically scan (line-to-line or plane-to-plane) by the surgical instrument display software displayed by the display device. Finally, as shown in step (G), the surgical instrument display software confirms whether the light sensing element reaches the position of the first feature point, the second feature point or the third feature point through the image capturing device.

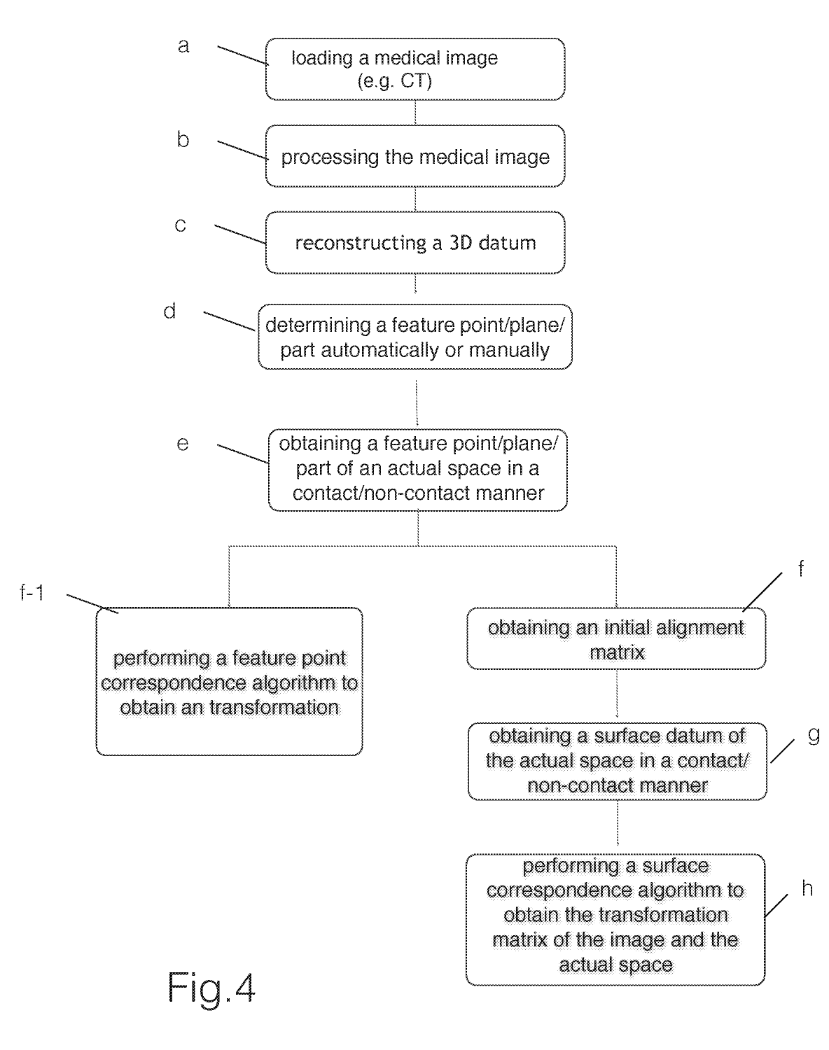

Next, FIG. 4 illustrates a registration way of physical characteristic information in the surgical instrument positioning method of the present invention. The calculation method in the surgical instrument display software comprise: (a) loading a medical image; (b) processing the medical image; (c) reconstructing a 3D datum; (d) determining a feature datum; (e) obtaining a feature datum of an actual space; (f) obtaining an initial alignment matrix or (f-1) performing a feature point correspondence algorithm to obtain an transformation matrix of the image and the actual space; (g) obtaining a surface datum of the actual space; (h) performing a surface correspondence algorithm to obtain the transformation matrix of the image and the actual space.

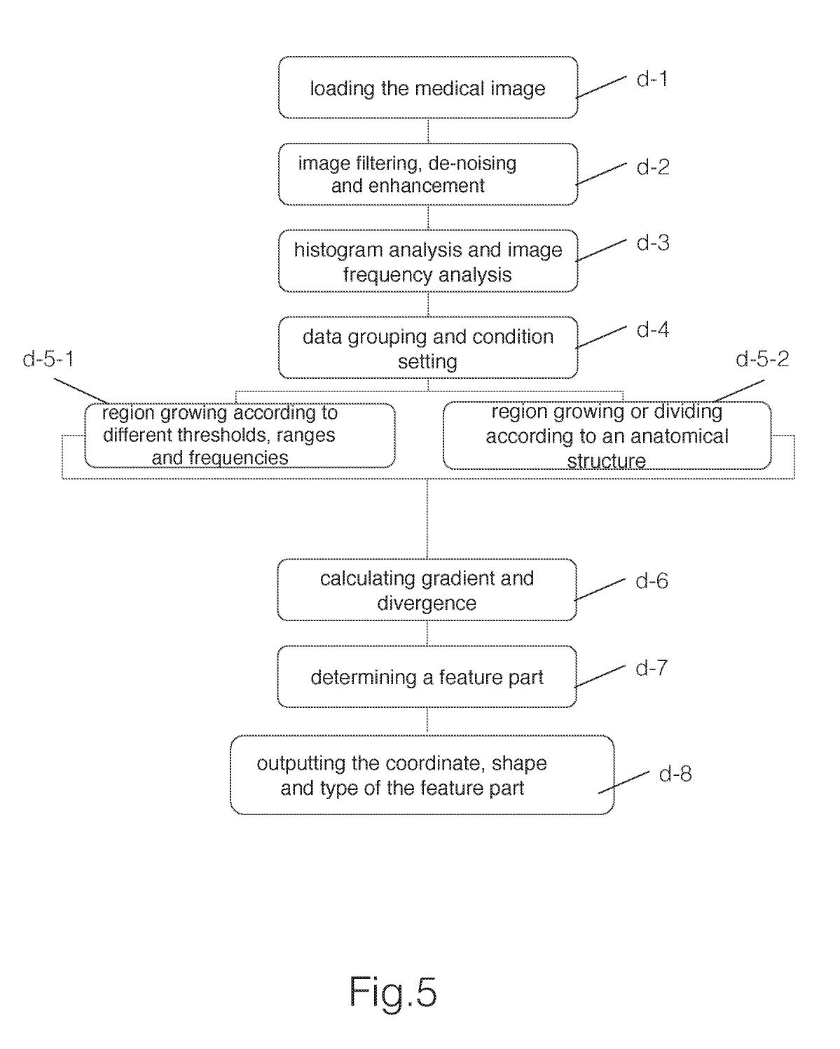

Next, FIG. 5 illustrates a flow chart of automatically determining a feature datum in the surgical instrument positioning method of the present invention. The flow is as follows: (d-1) loading the medical image such as CT; (d-2) image filtering, de-noising and enhancement to highlight specific lines or points or planes; (d-3) histogram analysis and image frequency analysis to confirm the characteristics of the points, lines, and planes; (d-4) data grouping and condition setting for grouping the characteristics of the approximate results; (d-5-1) region growing according to different thresholds, ranges and frequencies; (d-5-2) region growing or dividing according to an anatomical structure; (d-6) gradient and divergence calculation; (d-7) determining a feature part; (d-8) outputting the coordinate, shape and type of the feature part.

Furthermore, for the above-mentioned surgical instrument positioning method, the hand-held instrument is a surgical probe; the display device is a screen; the light sensing element is a reflective element; and the input device is a mouse or a touchpad. Wherein, the first feature point, the second feature point and the third feature point may be human body features, including a canthus, a nasal tip, an interdental gap, or a tooth socket. The first feature point, the second feature point and the third feature point may be established according to a human bone structure. The human bone structure may be a single tooth shape, a tooth line trend, or a tooth socket position.

In summary, the surgical instrument positioning system and the surgical instrument positioning method disclosed by the present invention are based on analyzing at least three positions of the surface image datum to be correspondingly set and recorded as a first coordinate datum, a second coordinate datum and a third coordinate datum. The first coordinate datum, the second coordinate datum and the third coordinate datum are established as a first feature point, a second feature point and a third feature point, respectively. Therefore, the first feature point, the second feature point and the third feature point can be set as absolute positions. When the electronic computer is equipped with the surgical instrument display software, the relative position of the real-time image position datum of the light sensing element can be calculated. In this way, there is no need for the operator to set a number of reflective elements at the surgical site, which will avoid the obstacles caused by the reflective elements during the surgical operation. In the case of tooth implantation, for example, the patient's head may need to be turned left and right. When the first feature point, the second feature point and the third feature point are set as the absolute positions, the relative position of the real-time image position datum of the light sensing element can be calculated by the electronic computer. The operator can arbitrarily move the patient's surgical site, which demonstrates the efficacy of the present invention.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A surgical instrument positioning system, used for confirming the position of a front end of a hand-held instrument relative to a human body when performing a surgical operation, the surgical instrument positioning system comprising:
a light sensing element, disposed on the hand-held instrument;
an image capturing device, configured to continuously or intermittently capture a surface image datum of the human body and a real-time image position datum of the light sensing element;
an electronic computer, electrically connected to the image capturing device for receiving the surface image datum and the real-time image position datum of the light sensing element, the electronic computer calculating the position of the hand-held instrument relative to the human body and the image position datum based on the position of the light sensing element, the electronic computer being equipped with a surgical instrument display software for analyzing at least three positions of the surface image datum to be correspondingly set and recorded as a first coordinate datum, a second coordinate datum and a third coordinate datum, the first coordinate datum, the second coordinate datum and the third coordinate datum being respectively established as a first feature point, a second feature point and a third feature point; and
a display device, electrically connected to the electronic computer for displaying the first feature point, the second feature point and the third feature point and the real-time position of the hand-held instrument or for displaying a simulated stereoscopic image of the hand-held instrument and its surroundings.

2. The surgical instrument positioning system as claimed in claim 1, wherein the first coordinate datum, the second coordinate datum and the third coordinate datum are absolute positions, and the real-time image position datum of the light sensing element is a relative position of the first coordinate datum and the second coordinate datum and the third coordinate datum.

3. The surgical instrument positioning system as claimed in claim 2, further comprising an input device electrically connected to the electronic computer, the input device being configured to select the first feature point or the second feature point or the third feature point by the surgical instrument display software displayed by the display device, the surgical instrument display software confirming whether the hand-held instrument on which the light sensing element is mounted reaches the position of the first feature point or the second feature point or the third feature point through the image capturing device.

4. The surgical instrument positioning system as claimed in claim 3, wherein the first feature point, the second feature point and/or the third feature point is a human body feature, including a canthus, a nasal tip, an interdental gap, a tooth line trend or a tooth socket.

5. The surgical instrument positioning system as claimed in claim 3, wherein the light sensing element is a reflective element or an active light emitting element.

6. A surgical instrument positioning method, used for confirming the position of a front end of a hand-held instrument relative to a human body when performing a surgical operation, the surgical instrument positioning method comprising the following steps:
(A) providing a light sensing element on the hand-held instrument;
(B) providing an image capturing device for continuously capturing a surface image datum of the human body and a real-time image position datum of the light sensing element;
(C) calculating the position of the hand-held instrument relative to the human body and the image position datum based on the surface image datum and the position of the light sensing element;
(D) providing a surgical instrument display software for analyzing at least three positions of the surface image datum to be correspondingly set and recorded as a first coordinate datum, a second coordinate datum and a third coordinate datum, the first coordinate datum, the second coordinate datum and the third coordinate datum being respectively established as a first feature point, a second feature point and a third feature point; and
(E) providing a display device for displaying the first feature point, the second feature point, the third feature point, and the real-time position of the hand-held instrument or a 3D simulation image.

7. The surgical instrument positioning method as claimed in claim 6, wherein the first coordinate datum, the second coordinate datum and the third coordinate datum are absolute positions, and the real-time image position datum of the light sensing element is a relative position of the first coordinate datum and the second coordinate datum and the third coordinate datum.

8. The surgical instrument positioning method as claimed in claim 7, after the step (E), further comprising the following steps: providing an input device for selecting the first feature point or the second feature point or the third feature point, the surgical instrument display software confirming whether the hand-held instrument on which the light sensing element is mounted reaches the position of the first feature point or the second feature point or the third feature point through the image capturing device.

9. The surgical instrument positioning method as claimed in claim 8, wherein the first feature point, the second feature point and/or the third feature point is a human body feature, including a canthus, a nasal tip, an interdental gap, a tooth line trend or a tooth socket.

10. The surgical instrument positioning method as claimed in claim 6, wherein a calculation method of the surgical instrument display software comprises:
(a) loading a medical image;
(b) processing the medical image;
(c) reconstructing a 3D datum;
(d) determining a feature datum;
(e) obtaining a feature datum of an actual space;
(f) obtaining an initial alignment matrix or (f-1) performing a feature point correspondence algorithm to obtain an transformation matrix of the image and the actual space;
(g) obtaining a surface datum of the actual space;
(h) performing a surface correspondence algorithm to obtain the transformation matrix of the image and the actual space.

11. The surgical instrument positioning method as claimed in claim 10, wherein the determining in the step (d) of the calculation method is an automatic determining or a manual determining.

12. The surgical instrument positioning method as claimed in claim 10, wherein the feature datum in the step (d) of the calculation method is a point, a plane or a part.

13. The surgical instrument positioning method as claimed in claim 10, wherein the feature datum of the actual space in the step (g) of the calculation method is obtained in a contact manner or a non-contact manner.

14. The surgical instrument positioning method as claimed in claim 11, wherein the process of automatically determining the feature datum comprises:
(d-1) loading the medical image;
(d-2) image filtering, de-noising and enhancement;
(d-3) histogram analysis and image frequency analysis;
(d-4) data grouping and condition setting;
(d-5-1) region growing according to different thresholds, ranges and frequencies;
(d-5-2) region growing or dividing according to an anatomical structure;
(d-6) calculating gradient and divergence;
(d-7) determining a feature part;
(d-8) outputting the coordinate, shape and type of the feature part.
